(19) United States
(12) Patent Application Publication
(10) Pub. No.: US 2011/0045482 A1
(43) Pub. Date: Feb. 24, 2011

(54) SCREENING METHODS FOR IDENTIFYING TARGET ANTIFUNGAL GENES AND COMPOUNDS BY DETECTING CELL SURFACE GLYCOPROTEINS

(75) Inventors: Peter Lipke, Brooklyn, NY (US); Marlyn Gonzalez, Forest Hills, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: RESEARCH FOUNDATION OF THE CITY UNIVERSITY OF NEW YORK, New York, NY (US)

(21) Appl. No.: 12/867,113

(22) PCT Filed: Feb. 10, 2009
(86) PCT No.: PCT/US09/33675

§ 371 Date: Sep. 20, 2010

Related U.S. Application Data
(60) Provisional application No. 61/065,505, filed on Feb. 11, 2008.

(51) Int. Cl. C12Q 1/68; G01N 33/53

(52) U.S. Cl. 435/6; 435/7.1

(57) ABSTRACT

The present invention relates to an assay method that can be used for high-throughput detection of cell surface glycoproteins. Specifically, the secretion of a chimeric glycoprotein reporter signals disruption of GPI anchor-mediated attachment of the glycoprotein to the cell surface. This method provides a high signal-to-noise ratio and is particularly useful for screening compounds that affect GP1 anchor biosynthesis. The method of the present invention thus permits genome-wide screens for genes that are needed for GPI anchor-mediated attachment of a glycoprotein to the surface of a cell as well as chemical inhibitors of proteins that promote GP1 anchor-mediated attachment of a glycoprotein to the surface of a cell. Protein inhibitors identified by the present method could be useful in antifungal drug treatments as well.

This application claims the benefit of priority to U.S. Provisional Patent Application No. 61/065,505, filed Feb. 11, 2008, which is hereby incorporated by reference in its entirety.

### FIELD OF THE INVENTION

The present invention generally relates to high throughput assay formats that facilitate detections of cell surface glycoproteins, and that are well suited for rapid and sensitive detections of genome-wide GPI anchor-mediated extracellular glycoprotein attachment.

### BACKGROUND

Cell surface glycoproteins are poised to regulate numerous cellular processes including immune defense, viral entry, parasitic infection, cell-cell adhesion, and signal transduction. The attachment of glycoproteins on the cell surface is required for maintaining endogenous cellular function, and disruption of extracellular glycoprotein attachment can interfere with critical cellular events.

Glycoproteins are often attached to the cell surface via a glycosylphosphatidylinositol (GPI) anchor assembly. GPI anchored proteins, eluding host-pathogen binding proteins such as adhesins, are essential structural and functional components of fungal cell walls. GPI anchored proteins have been found in species ranging from bacteria to invertebrates and vertebrates. GPI-anchored proteins are ubiquitous throughout the animal kingdom and play important roles in the orchestration of host-pathogen interactions during the infective process.

Currently, there is a pressing need for genome-wide functional assays for profiling the function of fungal cell wall genes. The few previously described assays that have been successful in identifying cell wall deficiencies fail to identify specific-cellular processes or reveal relevant genes involved in cell wall biosynthesis. Existing screens are primarily directed to drug-sensitivity assays and assays to characterize the sensitivity of target cells to toxic substances such as caffeine or the detergent SDS. An exception has been described where sensitivity to the cell wall perturbants congo red, calcoflour white, and killer toxin identified genes involved in synthesis and processing of cell wall polysaccharides (Pagé et al., Genetics (2003) 163(3): 875-94). Although such assays were carried out at large scale, they did not encompass the entire genome and thus may not have identified many genes that are important thr cell wall development. Genome-wide surveys for genes encoding GPI-mannoproteins have been done in silico (De Groot et al., Yeast (2003) 20(9):781-96.), but such assays are limited by the extent and quality of experimental data available. Mass spectrometry has also been used previously as a tool to identify and quantify cell wall GPI-protein; this approach has been applied to normal cells, but has only been extended to a few cell will mutants because mass spectrometry is not amenable to high throughput screening (Yin et al., J Biol Chem (2005) 280(21):20894-901).

### SUMMARY OF THE INVENTION

The method of the present invention can be used to detect reporter-modified glycoproteins, which can further be used to identify target genes that are needed for GPI anchor-mediated attachment of a glycoprotein to the surface of a cell and/or to identify important protein inhibitors (i.e., chemical inhibitors of proteins that catalyze GPI anchor-mediated attachment of a glycoprotein to the surface of a cell). Protein inhibitors identified by the present method could be useful in antifungal drug treatments as well.

In one embodiment, the present invention relates to a method for detecting reporter-modified glycoprotein secreted from cells in culture, comprising the steps of: obtaining a plurality of cells that express one or more GPI-anchored proteins; trans ruling the cells in parallel with a plasmid expression vector comprising a gene sequence encoding a reporter-modified glycoprotein, wherein the reporter is a signal generating compound; incubating the transformed cells in culture media containing one or more osmoprotectants; isolating the culture media from the incubated cells; and detecting reporter-modified glycoprotein secreted into the isolated culture media.

In another embodiment, the present invention relates to a method for identifying genes required for GPI anchor-mediated attachment of a glycoprotein to the surface of cells in culture, comprising the steps of obtaining a plurality of cells, each containing a different gene deletion; transforming the cells in parallel with a plasmid expression vector comprising a gene sequence encoding a reporter-modified glycoprotein, wherein the reporter is a signal generating compound; incubating the transformed cells in culture media containing one or more osmoprotectants isolating the culture media from the incubated cells; detecting reporter-modified glycoprotein secreted into the isolated culture media; and identifying genes required for GPI anchor-mediated attachment based on the amount of reporter-modified glycoprotein detected.

In yet another embodiment, the present invention relates to a method for identifying chemical inhibitors of proteins that promote GPI anchor-mediated attachment of a glycoprotein to the surface of cells in culture, comprising the steps of, obtaining a plurality of cells; transforming the cells in parallel with a plasmid expression vector comprising a gene sequence encoding a reporter-modified glycoprotein, wherein die reporter is a signal generating, compound; combining the transformed cells with culture media containing one or more osmoprotectants, and adding a different known chemical inhibitor to each cell culture; incubating the cell cultures; isolating the culture media from the incubated cells; detecting reporter-modified glycoprotein secreted into the isolated culture media; and identifying chemical inhibitors of proteins that promote GPI anchor-mediated attachment of a glycoprotein to the surface of cells in culture based on the amount of reporter-modified glycoprotein detected.

The plurality of cells includes two or more cells. For us in high-throughput screening, the plurality can include several hundred cells or even more, as may be found in a commercially available library of cells encompassing an entire genome. One or more cells may be derived from a fungal species, such as Candida albicans, Aspergillus fumigatus, Ustillago maydis, Cryptococcus neoformans, and Schizosaccharomyces pombe, and combinations thereof. The cells may be derived from other species of microorganisms as well. In one embodiment, the reporter is GFP. In another embodiment, the cells are incubated at a temperature of 15° C. to 20° C. for 1-3 days, preferably 18° C. and preferably for 2 days. In one embodiment, the osmoprotectant is sorbitol. In another embodiment, the plasmid expression vector is p416MG3. In yet another embodiment, the glycoprotein is selected from UPI-mannoprotein and an adhesin. The method of the present invention may also include the step of measuring the amount of detected reporter-modified glycoprotein and/or dissociated reporter. The amount of detected reporter-modified glycoprotein and/or dissociated reporter may be measured by various means, preferably by fluorimetry, immunoblot analysis, or both.

The present invention also provides a method of screening for novel antifungal drug targets. By comprehensively identifying genes required for fungal cell wan development, the method helps identify cell wall targeted antifungals. More specifically, the screening method of the present invention identities genes required for linkage of glycoprotein to a cell wall via a GPI anchor. To our knowledge, no such genome-wide screening approach has been previously published. The screening approach described herein is applicable, to any species of microorganism with GPI anchored surface proteins, including various pathogenic fungal species, such as Candida albicans and other members of the genus Candida, and Aspergillus fumigatus and other members of the genus Aspergillus, and other fungi that may be shown to have GPI-anchored cell wall proteins, including Cryptococcus neoformas, Schizosaccharomyces pombe, and plant-parasitic fungi such as the agents of pima infections like smut (Ustillago maydis) and wheat rust. In addition to being useful in pinpointing novel cell wall targets for drug design, the screening method of the present invention can be conveniently adapted as a high-throughput antifungal drug screen.

As described herein, the inventive assay relies on the endogenous machinery of the cell to install an engineered reporter-modified glycoprotein on the cell surface. Specifically, when the proteins from the GPI anchor biosynthetic pathway are disrupted by gene mutation or are in the presence of chemical inhibitors, the reporter-modified glycoprotein is secreted into the growth medium to an abnormal extent. The secreted reporter modified glycoprotein then serves as the readout for the assay and its presence can be monitored spectroscopically. In the present invention, it was surprisingly found that cell cultures grown under the conditions of the present invention (particularly at temperatures lower than standard incubation temperatures) led to a high yield of secreted reporter-modified glycoprotein that allows for detection and measurement in a new and meaningful way, particularly because prior methods did not result in high enough yields of secreted reporter-modified glycoprotein to be detectable apart from interference fluorescence from the cell culture media itself.

### BRIEF DESCRIPTION OF THE DRAWINGS

FIG. 1: Images of cell surface detection of Green Fluorescent Protein (GFP)-glycoprotein (A): Image of yeast cells transformed with a gene encoding a GFP-glycoprotein that show cell surface attachment of the reporter by fluorescence microscopy. (B): Image of the cell surface localization of GFP-glycoprotein confirmed on yeast cells by electron microscopy. The GFP-glycoproteins appear as black particles on the cell surface. (C): Depiction of cell surface attachment of GFP-glycoprotein.

FIG. 2: Schematic depiction of a high-throughput assay for detecting the secretion of a GFP-glycoprotein from cells, wherein a library of mutant cell strains is transformed with a vector comprising a gene insert that encodes a chimeric GFP-glycoprotein. Transformed cells are cloned in parallel on petri plates and selected samples are subsequently, collected and grown in culture. Secretion of the GFP-glycoprotein can be quantified by fluorimetry or by immunoblot analysis.

FIG. 3. Graph depicting fluorescence quantification of GFP-glycoprotein secreted from mutant samples. Samples of supernatant isolated from mutant cultures transformed with a GFP-glycoprotein gene construct were examined by fluorometry. The average florescence intensity (•) detected for each tested mutant sample is indicated. Error bars indicate standard deviation. The average fluorescence intensity for wildtype samples (••••••) and the associated standard deviation (----) is also shown. Data plotted without error bats were not replicated.

FIG. 4. Image from immunoblot analysis of GFP-glycoprotein secreted from mutant samples. Representative samples of supernatant isolated from mutant cultures transformed with a gene encoding a GFP-glycoprotein were examined by immunoblot analysis. Specifically, supernatant retrieved from mutant and wildtype cultures were applied to a nitrocellulose membrane and subsequently probed with an α-GFP antibody. Enhanced signal intensity with respect to wildtype indicates hyper-secretion of the GFP-glycoprotein. Hypo-secretion of the GFP-glycoprotein is indicated in samples that generate a faint signal.

FIG. 5. Schematic depiction of a high-throughput screen of mutant library genes involved in GPI-glycoprotein anchoring to cell wall, wherein a library of mutant cell strains is prepared in a 96-well microtiter plate format. Cell suspension aliquots from the library are collected and transformed with a gene expressing a GFP-glycoprotein. Mutants are incubated in culture and the presence of the GFP-glycoprotein in the supernatant can be detected using a florescence plate reader or by immunoblot analysis.

FIG. 6. Images from immunoblot analysis of GFP-glycoprotein present in supernatant isolated from wildtype (WT) cells and kre5 and cwp1 mutants. (A): Supernatant retrieved from WT, kre5 and cwp1 cultures were applied to a nitrocellulose membrane. The blot was treated with α-GFP antibodies thr detection of the GFP-glycoprotein in the supernatant. As indicated, supernatant samples isolated from kre5 and cwp1 mutants showed enhanced levels of the GFP-glycoprotein with respect to wildtype. Addition or sorbitol was shown to increase the presence of GFP-glycoprotein. (B): Prolonged exposure of an α-GFP immunoblot shows enhanced levels of GFP-glycoprotein in kre5 and cwp1 mutants and in the presence of sorbitol.

FIG. 7. (A): Schematic of reporter construct encoding pGFP-Sag1p. (B): Restriction analysis of empty plasmid (lane 1) and of pGFP-Sag1p (lane 2) with Spe1 and Sho1. Expected product sizes: 6.4 kb (lane 1) and 6.4 kb and 1.7 kb (lane, 2).

### DETAILED DESCRIPTION OF THE INVENTION

The present invention relates to methods for detecting the secretion of reporter-modified glycoproteins from cells in culture by using controlled disruption oldie biosynthetic pathway that governs GPI anchor-mediated glycoprotein attachment to the cell surface as a means for modulating critical cellular events. This method efficiently provides reproducible data that can be used to identify antifungal drug targets.

As used herein, the term “reporter” refers to a signal generating compound. GFP and color variants thereof are suitable reporter molecules that can be used in the present invention. Other suitable signal-generating compounds include, for example, chromagens, catalysts such its enzymes, luminescent compounds such as fluorescein and rhodamine, chemiluminescent compounds such as dioxetanes, acridiniums, phenanthridiniums and luminol, radioactive elements, and direct visual labels. Suitable enzymes include, for example, alkaline phosphatase, horseradish peroxidase, β-galactosidase, and the like, in another embodiment, α-galactosiadase can be used as the reporter molecule. The selection of particular label is not critical, but it must be capable of being connected to a glycoprotein and producing a signal either by itself or in conjunction with one or more additional substances.

As used herein, the term “reporter-modified glycoprotein” refers to a reporter, as defined above, that is fused to a glycoprotein.

As used herein, the term “glycoprotein” refers to a protein that contains one or more carbohydrate groups covalently attached to a polypeptide chain. Suitable glycoproteins include any protein that can be installed on the cell surface via a GPI anchor assembly, such as enzymes, antigens, and adhesin molecules. For example, the glycoprotein can be a mannoprotein, a protease of carbohydrate-active enzyme, or a “disguising antigen” that a pathogen uses to escape immune detection (e.g., Variable Surface Glycoproteins in the genus Trypanosoma).

The reporter molecule may be installed onto the target glycoprotein by conventional methods known to those of ordinary skill in the art. For example, the reporter-modified glycoprotein can be prepared as a fusion protein comprising GFP and the cell wall anchorage domain of a glycoprotein. That is, a gene can be synthesized as a chimera of a cell wall glycoprotein gene and a GFP gene, which can then be inserted into a vector module that is then inserted into cells in culture.

A gene encoding a fusion protein according to the present invention may be manufactured using standard recombinant DNA techniques. For example, DNA fragments coding tin the different polypeptide sequences can be ligated together in-frame in accordance with conventional techniques. For example, this can be accomplished by employing blunt ended or stagger-ended termini for ligation, using restriction enzyme digestion to provide for appropriate termini, filling in of cohesive ends as appropriate, and using alkaline phosphatase treatment to avoid undesirable joining and enzymatic ligation. In another embodiment, the fusion gene can be synthesized by conventional techniques, such as those including automated DNA synthesizers. Alternatively, PCR amplification of gene fragments can be carried out using anchor primers which give rise to complementary overhangs between two consecutive gene fragments that can subsequently be annealed and re-amplified to generate a chimeric gene sequence (see, e.g., Ausubel et al., CURRENT PROTOCOLS IN MOLECULAR BIOLOGY (1992) John Wiley & Sons, Inc,). Moreover, many expression vectors are commercially available that already encode a fusion moiety (e.g., a GFP polypeptide).

In one embodiment of the present invention, the reporter plasmid prepared by first constructing a GFP gene and a glycoprotein gene by PCR. The GFP and glycoprotein DNA and the DNA of a commercial plasmid, for example p416MG3 are then treated with a restriction endonuclease in order to produce intermediates with staggered and complementary termini for ligation. The intermediates can be combined by base paring and linked by action of a DNA ligase. The constructs can be verified by restriction analysis on an agarose gel and sequenced. The resulting plasmids can be propagated and purified using, for example, a commercial plasmid purification kit.

As used herein, the term “vector” refers to a nucleic acid molecule capable of transporting another nucleic, acid to which it has been linked. One type of vector is a “plasmid,” which refers to a circular double stranded DNA loop into which additional DNA segments can be ligated. Another type of vector is a viral vector, in which additional DNA segments can be ligated into the vital genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors or utility in recombinant DNA techniques are often in the form of plasmids. In the present specification, “plasmid” and “vector” can be used interchangeably as the plasmid is the most commonly used form of vector.

In the present invention, the vector may be a commercial vector, such as p416MG3 or other pRS vectors from American Type Culture Collection or any commercial expression vector such as the pYES or pYES-TOPO vectors (Invitrogen). Other suitable vectors will be known to those of ordinary skill in the art.

The vectors, of the present invention can be designed for expression of a reporter-glycoprotein in prokaryotic or eukaryotic cells. In one embodiment of the present invention, the reporter-glycoprotein genes can be expressed in fungal species such as Candida albicans, and other members of the genus Candida, and Aspergillus fumigatus and other members of the genus Aspergillus, and other fungi that may be shown to have GPI-anchored cell wall proteins, including Cryptococcus neoformans, Schizosaccharomyces pombe, and plant-parasitic fungi such as the agents of plant infections like smut (Ustillago maydis) and wheat rust. In principle, any test strain or set of strains can be used. In another embodiment, cell strains can be transformed on a large scale by conventional means, such as by using the EZ-yeast transformation kit available (torn MP Biomedicals. A single kit can enable transit nation of about 200 different strains in less than 3 hours.

In one aspect of the present invention, high-throughput transformation of gene deletants can be accomplished using the bio101/EZ-yeast transformation kit designed for large scale transformation of yeast. 100 transformations can be performed simultaneously in about 3 hours wherein preparation of competent cells is not required. Cells for transformation can be obtained from fresh growth patches in sterile 127.8×85.5 mm rectangular petri dishes (Fisher Scientifics). Approximately 2-3 min cell dumps can be isolated for each individual strain using sterile wood sticks and transferred to 125 μL of transformation mix buffer, previously added to wells of a sterile 96-well microtiter plate. In one embodiment, about 2 μg of transforming/plasmid DNA (pGFP-Sag1p) and about 5 μL of EZ-yeast carrier DNA can be added to cell suspensions in the wells. The 96-well microtiter plate can be gently shaken and incubated at, (hr example, 30° C. for 30 min. Following incubation, the entire content of each well can be plated in 10 cm×10 cm petri plates in plasmid selective media containing geneticin to which gene deletants are resistant. Cell spreading on transformant-selection plates can be performed by addition of sterile 5 mm glass beads into the plate with swirling the plates either by hand or by use of a rotating platform.

In one aspect of the invention, 3 mL cultures of transformed colonies (of similar size) are grown at 18° C. in 13×100 mm borosilicate tubes in plasmid-selective medium (complete minimum medium lacking uracyl) containing 1 M sorbitol and buffered to pH 6.5 with the biological buffer MOPS. Growth in small test tubes permits easy monitoring of cell growth by monitoring optical density, measurements at 669 nm from the tubes directly. The cultures may be incubated using a set up that allows for simultaneous growth of, for example, 100 3 cultures. The cultures are grown using, the parameters recited herein under which GFP reporter protein hyper-secretion was observed for cell wall mutants used as positive controls (cwp1/cwp1, kre1/kre1 and KRE5/kre5). To account for differences in growth rate among mutants, OD readings are performed regularly. Cultures that exhibit an optical density within the range of, for example, 0.5-0.6 at 660 nm are centrifuged, and 500 μL of the resulting cell-free supernatant may be stored at −80° C. in silicon-coated tubes in the presence of fungi-specific protease inhibitor cocktail (Sigma). In the present invention, it has been found that these storage conditions provide the least protein loss. The stored supernatants can then be thawed on ice and assayed for GFP fluorescence by single wavelength fluorescence spectroscopy using a 96-well microliter plate suitable for fluorescence measurements.

The reporter gene may be expressed wider regulation of a promoter that drives expression of a gene essential for cell survival. It is predicted that genes that are essential for cell survival exhibit the least noise in expression levels or are under much tighter regulation because of the essential role that they play in the cell (Fraser et al., PLoS Biol. (2004) 2(6): p. e137). For example, the promoter of glyceraldehydes-3-phosphate dehydrogenase (GPO) from Saccharomyces cerevisiae may be used. The GPO promoter is a strong constitutive promoter in Saccharomyces cerevisiae, and normally promotes expression of the glycolytic enzyme glyceraldehyde-3-phosphate dehydrogenase. Any promoter that drives expression of a gene essential for cell survival is within the scope of the present invention. Phosphoglycerate kinase (PGK) is another example of a suitable promoter. Regulatable promoters such as GAL1 or CUP 1 of Saccharomyces cerevisiae, or other suitable inducible or constitutive promoters specific to other organisms, such as the repressible TET promoter, can also be used.

As used herein, the terms “cell culture” and “cells in culture” refer to any in vitro culture of cells. Included within this term are bacterial and fungal species and strains, protozoa with GPI anchored proteins such as trypanosomes or plasmodia, continuous cell lines (e.g., with an immortal phenotype), primary cell cultures, transformed cell lines, finite cell lines (e.g., non-transformed cells), and any other cell population from any taxon that has GPI anchors maintained in vitro. Such taxa will include animals, plants, protozoa, and archaea. According to the present invention, cells in imitate may be grown in an aqueous environment wherein the culture medium comprises the physiochemical and nutritional requirements for survival and growth of the cells. Cell culture conditions will vary for each cell type used and methods for determining optimal conditions for cell culture are within the grasp of those of ordinary skill in the art.

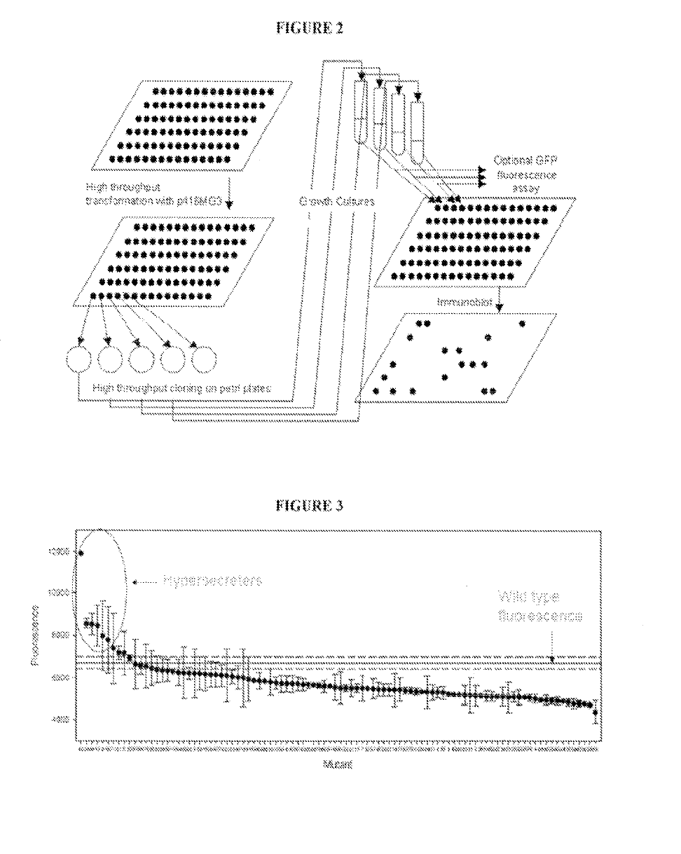

In one embodiment of the present invention, an osmoprotectant, such as sorbitol, can be added to the growth media. The present inventors have discovered that sorbitol provides a cellular environment that is osmotically stable. Specifically, sorbitol raises the solute concentration of the media to correlate with concentration levels present inside die cell, and thus prevents osmosis-mediated flux of water into the cells. In wildtype cells, the cell wall acts as a barrier that prevents deleterious osmosis. In mutant cells wind possess compromised cell walls, osmosis will cause the cell to fill with water, causing the cell to eventually burst. Addition of sorbitol was surprisingly discovered to prevent cell bursting. Sorbitol was also unexpectedly found to enhance protein yield. Other osmoprotectants including, for example, glycerol, trehalose, other saccharides, insulin, or salts are expected to have the same effect. As depicted in FIG. 2, cells grown in the presence of sorbitol display increased levels of GFP in the supernatant. It was also observed that sorbitol induces production of glycerol inside the cell. Glycerol promotes protein folding, reduces protein degradation, and may indirectly increase protein yield. It is contemplated that an increased yield of soluble GFP observed in the supernatant isolated according to the present invention results from enhanced protein folding as unfolded protein is quickly degraded inside the cell.

In one embodiment of the present invention, cells are grown in culture in media that is buffered at a pH of about 65. The full range of pH values is accessible as appropriate for the specific culture or cell line, from pH 0 to pH 14 if GFP is to be detected using anti-GFP antibodies. If fluorescence is to be measured directly, due to sensitivity of the GFP fluorophore to acidic and highly alkaline conditions, a suitable pH would be in the range of 4-9. For example, a cell culture of Saccharomyces cerevisiae may be carried out at a low pH of about 4.0. GFP fluorescence is susceptible to acidic conditions because fluorescence is irreversibly destroyed due to fluorophore reduction (Tsien, Annu. Rev. Biochem., (1998) 67:509-44). Yeast cells acidify media during growth and thus produce culture conditions that destroy fluorescence. The present inventors have discovered that buffering the media with a biologically tolerated butler (e.g., MOPS) to a particular pH (e.g., about 6.5 for Saccharomyces cerevisiae) provides a growth environment that promotes proper folding and oxidation of the GFP fluorophore, thereby maximizing the likelihood that GFP molecules will remain fluorescent. Suitable buffers and appropriate pH ranges can be identified for other species, either from the literature or experimentally. In this embodiment, any growth pH between pH 3 and pH 11 will allow native conformation and fluorescence of the current version of GFP. If the pH value is between pH6 and pH9, the GFP will fold autologously, and fluorescence may be directly assayed. If the growth is outside of this range, then the fractions to be assayed will need to be adjusted to within pH6 to pH 9 to promote folding before fluorescence assay. The immunoassay embodiment is effective across all possible growth pH values. This, in turn, minimizes variability in GFP levels between different mutants and clones of the same mutant. Any cell culture buffer that can maintain the pH of than media to about 6.5 can be used in the present invention, in one embodiment. MOPS buffer is included in the culture media for Saccharomyces cerevisiae at a concentration of about 0.165 M, buffered at a pH between pH 6.2 and pH 8.2. Any non-toxic buffer can be used at its suitable pH range and concentration, including TRIS, carbonate/bicarbonate, MES, PIPES, phosphate, succinate, citrate and the like. Other buffers and concentrations may be determined from the literature or experimentally.

It was also surprisingly discovered that cell cultures grown at low temperatures (e.g., preferably 18° C. for Saccharomyces cerevisiae) rather than standard temperatures led to higher reporter protein yields, possibly because culturing at low temperature ma promote protein folding of molecules that are otherwise be destroyed in their unfolded state. Temperatures sub-maximal for growth consistently increase yields and decrease variability of the assay. In the present invention, cells may be cultured at any suitable temperature between about 5° C. to about 60° C., preferably about 15° C. to about 22° C. for Saccharomyces cerevisiae. Other temperatures for other organisms may be determined as that yielding maximal fluorescence and/or reproducibility of fluorescence in a specific embodiment.

The present invention is not limited to any particular culture system. Indeed, a variety of culture systems may be used, including, but not limited to roller bottle cultures, perfusion cultures, batch fed cultures, petri dish cultures, and multi-well (e.g., 96-well) culture plates.

Cell growth can be monitored, for example, by using spectroscopic optical density measurements. Different stages of the cell cycle can be targeted by measuring the optical density of the cells in culture. In one embodiment of the present invention, optical density measurements are used to ascertain an optimal stage for supernatant collection. The optical density of cells in culture is measured and, after reaching the desired level. (To measure GFP secretion levels in cell-free supernatants, the cells are grown to logarithmic phase, achieving, for example, an optical density of about 0.5-0.6 when measured at 660 nm. It was observed herein that stationary phase cultures (with an optical density within the range of about 0.5 at 660 nm) exhibited as substantial disappearance of the GFP reporter protein in culture. In a preferred embodiment of the present invention, the cells are centrifuged for as short a period as for complete removal pelleted cells from the culture medium. En one embodiment of the present invention, the cells are centrifuged for about 10 minutes. In one embodiment, the supernatant is then assayed for reporter-modified glycoprotein levels, which can be quantified.

Reporter-modified glycoprotein levels may be detected and measured in the supernatant using various methods, such as spectroscopically—i.e. using die analytical technique of directing incident light at a designated sample, reading the light following its interaction with the sample, and then making a determination of the contents of the sample based upon measured differences between the incident light and the detected light. In one embodiment, the amount of GFP-glycoprotein secreted in the supernatant can be detected and quantified by measuring the fluorescence, of the supernatant.

Reporter-modified glycoprotein levels may also be detected in the supernatant by immunoblot analysis. As used herein, the term “immunoblot analysis” refers to an analytical technique used to detect specific proteins in a given sample. Immunoblot analysis uses gel electrophoresis to separate proteins based on the relative mass to charge ratio of the polypeptides. The proteins are then transferred to a protein-binding membrane where they are detected using antibodies specific to the target protein. In the present invention, the supernatant may be applied to a protein-binding membrane and the presence of the reporter-protein construct can be detected on the membrane with a reporter-specific antibody. Suitable protein-binding membranes include, for example, cellulose-based membranes such as nitrocellulose. In one embodiment, the levels of a GFP-glycoprotein construct in the supernatant can be detected by transferring an aliquot of the supernatant to a nitrocellulose membrane followed by treatment with an α-GFP antibody.

As disclosed herein, immunoblot analysis and fluorimetry are complimentary approaches for sensitive and high-throughput detection of the reporter protein. Fluorescence facilitates fast and reproducible detection. Immunoblotting is self-consistent, and sensitive to degree of glycosylation present on the reporter protein (which can be assessed by blotting to membrane vs. blotting to membrane coated with concanavalin A) as well as protein folding and other factors.

Fluorescence and immunoblot analysis are highly efficient means for detecting the reporter-glycoprotein in the supernatant. Both detection methods are amenable for high throughput analysis. Direct fluorescence measurements are preferred.

The method of the present invention provides a genome-wide screen that can complement known experimental data retrieved from computer-based surveys and can significantly contribute to the discovery of genes involved in fungal cell wall development. The genome-wide strategy presently disclosed has been specifically designed to handle systematic growth of hundreds of strains at a time. Hyper-secretion and hypo-secretion of the marker (reporter) is reproducibly assayed 96-well plates. Therefore, the procedure is easily scalable and adaptable for screening a range of fungi or other species and under a variety of growth conditions. Importantly, the method of the present invention facilitates determination of growth stage by reading optical density directly from the test tubes that contain cultured cells. In one embodiment, the test tubes containing the cell culture are inserted directly into a spectrophotometer to obtain concentration readings. This is a convenient feature that allows one to assay for GPI-protein attachment to the cell surface at any stage of the cell growth cycle. Gene deletion strains expressing a reporter-modified glycoprotein can be grown in small test tubes of about 3 mL, or at any volume suitable for spectrophotometry. For example, strains can be grown in 0.1 mL cultures in a microtiter plate. Depending on the organism, any growth temperature and growth time is practicable between 0° C. and 45° C., and from a few hours to several weeks. That is, cells can be cultural in growth media for any time period needed to achieve exponential growth phase. For individual species, suitable growth times can be experimentally determined based on the time of growth needed to give maximal GFP production and reproducibility. For instance, Saccharomyces cerevisiae cells can be grown in culture until achieving an optical density of about 0.5 to about 0.6 at 660 nm at a growth temperature of about 18° C.

The present invention also enables the identification of deficiencies in glycoprotein linkage to the cell wall through GPI anchors. Such deficiencies may, for example, result from mutations in the genes necessary for proper assembly of the cell wall, or front interference in cell wall assembly with chemical inhibitors. Disrupted GPI anchor biosynthesis promotes secretion of the reporter-modified glycoprotein into the growth medium at levels that are higher (hyper-secretion) or lower (hypo-secretion) than the wild type parental or untreated strains.

The present method uses a chimeric reporter GPI-glycoprotein, made by fusing the cell wall anchorage domain of a glycoprotein with a reporter, such as GFP. Previous experiments have shown that fungal cells unable to properly anchor such glycoproteins to the wall secrete these proteins into the growth medium at higher levels than those observed for normal cells. (Wojciechowicz et al., Mol. Cell. Biol. (1993) 13: 2554-63; Lu et al., Mol. Cell. Biol. (1994) 14: 4825-33; Lu et al., J. Cell Biol. (1995) 128: 333-40; van der Vaart et al., FEMS Microbiol Lett. (1996) 145(3):401-07). Such deficiencies result from mutations in genes involved in the attachment of these glycoproteins to the fungal wan. Test data was generated from as commercially available gene deletion library for Saccharomyces cerevisiae (from Invitrogen). In principle, any test strain or strain or set of strains can be used.

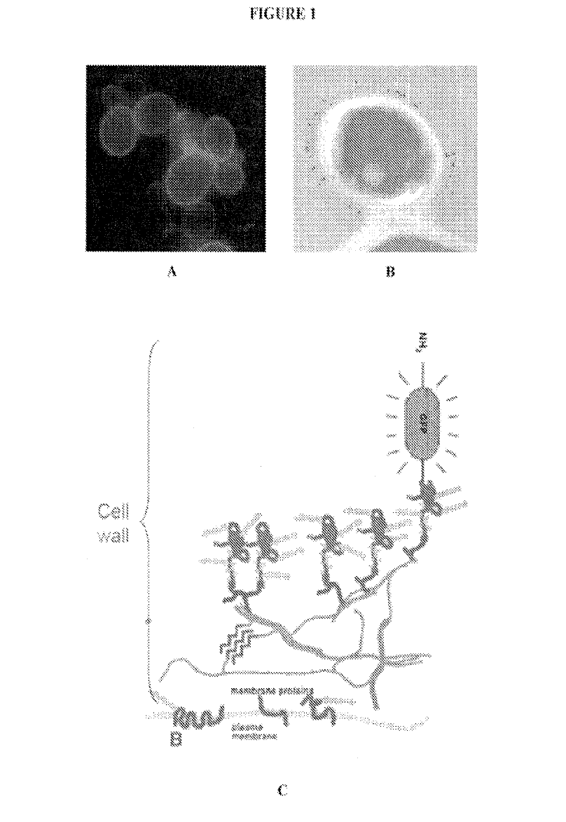

The S. cerevisiae genome contains about 6,000 genes. In one embodiment of the present invention the genes in the S. cerevisiae genome are individually deleted to generate about 6,000 different gene deletion strains forming a mutant library. The present invention enables screening of such to library for genes required for proper cross-linking of a GFP-glycoprotein reporter to the cell wall. The high-throughput approach of the present invention requires that each strain in the library expresses a gene encoding a GFP-labeled cell wall protein. For example, a reporter-glycoprotein gene can be synthesized as a chimera of a cell wall gene, and then GFP can be inserted into a vector molecule such as p416MG3, which can in turn be inserted into each strain of the library. This process may be performed on a large scale. For example, an EZ-yeast transformation kit available from MP Biomedicals may be used. A single kit can enables transformation of about 200 different strains in less than 3 hours. Cells harboring the artificial gene can be assayed for proper processing and localization of the fluorescent protein product. Expression and localization of the fluorescent protein to the cell wall can be confirmed by, for example, fluorescent and immunoelectron microscopy (FIG. 1). Methods for high-throughput analysis of secretion levels of the fluorescent cell wall protein are described according to the examples below and are illustrated in FIG. 2.

The method of the present invention can also be used to screen for drugs that are active against organisms with GPI anchored surface proteins. For example, malaria and trypanosomes are disease-causing organisms with GPI anchored surface proteins and could be targeted according to the present invention. Specifically, the inventive assay can be used to identify small molecule inhibitors that target specific proteins involved in GPI-anchor biosynthesis in the pathogenic cells. These cells could then be transformed with a reporter-modified glycoprotein and then incubated in culture with a library of putative chemical inhibitors that disrupt GPI-anchor biosynthesis, after which drug candidates could be identified as corresponding to the inhibitors that led to detectable levels of reporter-modified glycoprotein secreted into the culture.

### EXAMPLES

The present invention is next described by means of the following examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scone of equivalents to which the claims are entitled.

### Example 1

### Fabrication of GFP-Adhesin Gene Construct and Fungal Strain Transformation

(A) A plasmid fusion construct, pGFP-Sag1p, was constructed as follows: an EcoRI-BgIII fragment of the yeast enhanced GFP (yEGFP) gene from Aequorea victoria was prepared by PCR using pMut3-yEGFP as the PCR template (pMut3 was obtained from Yale University). A BgIII-XhoI fragment encoding the last 300 residues at the C-terminus of the cell wall GPI-glycoprotein, α-agglutinin, was similarly prepared using pH27 as the PCR template. A SpeI-EcoRI fragment encoding the invertase secretion signal and cleavage site was synthesized using overlapping oligomers of 51 and 53 base pairs each. Extension of the non-overlapping regions was achieved using Klenow DNA polymerase I Large Fragment New England BioLabs). Table 1 lists all the oligonucleotides used in this work. Each fragment was subjected to restriction digestion with the indicated enzymes. Restriction fragments were subjected to agarose gel electrophoresis and recovered from the gel using QIAquick Gel Extraction Kits (Fisher Scientifics). A four-fold ligation reaction involving all 3 restriction fragments described above and vector p416GPD1 (ATCC, Mannasas, Va., USA) bearing SpeI-Xho1 sticks ends was performed using T4 DNA Ligase (New England BioLabs). Constructs were verified by restriction analysis in 1.0% agarose gels and sequenced to exclude possible PCR artifacts. No mutations were found that would affect the amino acid sequence of the fusion constructs. The resulting plasmids were propagated in and purified from E. coli using a Qiagen plasmid purification kit according to the instructions of the manufacturer.

(B) A gene encoding a chimeric GFP-GPI α-agglutinin was prepared as follows: a fusion of the promoter sequences from yeast invertase, yeast adapted Green Fluorescent protein, and the C-terminal 300 residues of Sag1p was prepared. A fungi-optimized GFP cDNA was fused upstream of the last 900 base pairs of the S. cerevisiae SAG1 cDNA. SAG1 encodes the cell wall GPI-mannoprotein α-agglutinin. The signal sequence and cleavage site of the cell surface enzyme, invertase, was generated using overlapping oligomers and inserted upstream of the GFP cDNA. The resulting recombinant gene was inserted in front of the GPD1 promoter in CEN plasmid p416GPD1. The authenticity of the construct was confirmed by DNA restriction analysis as shown in FIG. 7B, and by sequencing. The arrangement of individual components of the reporter gene is shown in FIG. 7A. Table 1 below shows the oligonucleotides used for the construction of GFP-Sag1p. The synthesized gene was then inserted into a p416MG3/pGFP-Sag1p plasmid using conventional techniques.

TABLE 1

| | Length | | |
|---|---|---|---|
| Primer | (bp) | Length Sequence (5′→3′) | Remarks |
| InvP1 | 51 | GCGCCGACTAGTATGCTTTTGCAAGCTTTCCT | Underlined SpeI and overlapping |
| | | TTTCCTTTTGGCTGGTTTT | region |
| InvP2 | 53 | CGCCGGGAATTCTGATGCTGATATTTTAGCT | Underlined ECORI and |
| | | GCAAAACCAGCCAAAAGGAAAA | overlapping region |
| GFPP1 | 30 | GCGCCGGAATTCAGTAAAGGAGAAGAACTT | Forward primer, underlined ECORI |
| GFPP2 | 30 | CGCCGCAGATCTGTATAGTTCATCCATGCC | Reverse Primer, Underlined BglII |
| α-aggGPIP1* | 30 | GCGGCGAGATCTAGTGCGGTATTCCACTGGA | Forward primer, underlined BglII |
| α-aggGPIP2 | 30 | CGCCGCCTCGAGTTAGAATAGCAGGTACGA | Reverse primer, underlined XhoI |
| | | | and stop codon |

### Example 2

### High-Throughput Culturing of Transformed Fungal Cell Strains

A 6,000-membered mutant library comprising mutant strains of S. cerevisiae was purchased from Invitrogen. Each member of die library contained to single gene deletion encompassing the full S. cerevisiae genome of 6,000 genes. The library was prepared in 96 well microtiter plates wherein each well contained a single mutant.

Cell suspension aliquots from 76 mutant samples from the library was transferred to rectangular petri plates for culturing, as shown in FIG. 3. Additional strains were similarly prepared, but are not shown in FIG. 3. Colonies were grown using a high throughput plate replicator (Fischer model) as illustrated in FIG. 5. Each mutant was transformed using the reporter gene expressing the GFP-GPI mannoprotein referenced in Example 1. The transformation procedure was performed using commercial reagents provided in the “EZ-Yeast transformation kit” according to the manufacture's instructions (MP Biomedicals).

High-throughput transformation of gene deletants was accomplished using the bio101 EZ-yeast transformation kit designed for large scale transformation of yeast. 100 transformations were done simultaneously in about 3 hours. Typical overnight preparation of competent cells was not required thus substantially reducing processing time. Cells to be transformed were obtained from fresh growth patches in sterile 127.8×85.5 mm rectangular petri dishes (Fisher Scientifics). Approximately 2-3 mm cell clumps were selected for each individual strain using sterile wood sticks and transferred to 125 μL of transformation mix buffer, previously added to wells of a sterile 96-well microtiter plate. Following, 2 μg of transforming/plasmid DNA (pGFP-Sag1p) and 5 μl of EZ-yeast carrier DNA were added to cell suspensions in the wells. The 96-well micro plate was gently shaken and incubated at 30° C. for 30 min. Following incubation, the entire content of each well was plated in 10 cm×10 cm pent dishes in plasmid selective media containing geneticin to which gene deletants are resistant. Cell spreading on transformant-selection plates was performed with sterile 5 mm glass beads into the plate and swirling the plates either by hand in a rotating platform. The glass heads were recycled for net use.

Mutants were grown in a culture media that only permits growth of cells comprising the transformation vector.

In the specific, embodiment used as proof of principle for the present invention, homozygous and heterozygote diploid deletion strains in the BY4743 background, isogenic to the sequenced strain S288c, were used for viable and lethal deletions, respectively. All yeast strains used in this study were constructed during the EUROFAN project and obtained from the Invitrogen collection. All strains, apart from the deleted gene share the genotype; MATa/α his3Δ1/his3Δ1, leu2Δ/leu2Δ, lys2Δ/LYS2, MET15/met15Δ, ura3Δ/ura3Δ. Yeast was grown in either YPD medium (1% yeast extract, 2% peptone, 2% glucose) or defined medium (0.2% yeast nitrogen base without amino acids, 0.5% ammonium sulfate, 2% glucose, and 0.08 complete synthetic media (CSM) lacking uracyl,), containing 1 M sorbitol and buffered to pH 6.5 with 165 in M. MOPS and supplemented with 200 μM geneticin (Sigma Co.). Other suitable growth media may be used, as determined from the literature or experimentally.

Successful incorporation of the reporter gene was therefore accessed upon analysis of cell density over time. Transformed colonies of similar site were used to grow 3 mL cultures to achieve an optical density within the range of 0.5-0.6 at 660 nm at 15-18° C. Cells were cultured in media containing, CSM-URA (available from MP Biomedicals) and sorbitol (1 M) at pH 6.5). The corresponding cell-free supernatants were then assayed for GFP levels using a 96-well plate-reading fluorimeter as illustrated in FIG. 5. Transformants were incubated for 2 days at 18° C. in 3 mL of a growth media comprising sorbitol (1 M), and MOPS buffer (0.165 M) at 6.5. Cell density was monitored spectroscopically over time.

### Example 3

### High-Throughput Detection of Secreted GFP-Adhesin by Fluorimetry

Cells were cultured and transformed as in Example 2. Replicates of each mutant sample were cultured in parallel and were grown to similar concentrations. Cell concentration was determined by reading the optical density of the cell culture directly from the test tube using a spectrophotometer. The cell suspension was centrifuged lot 10 minutes in order to pellet cells away from the supernatant. The supernatant was then collected and levels of the secreted GFP-mannoprotein was measured by fluorimetry. Mean fluorescence intensity and standard deviation between samples was quantified and plotted as shown in FIG. 3.

Eight mutant strains were identified from the screen that exhibited enhanced secretion of the GFP-GPI mannoprotein with respect to wild type. Three of these mutants exhibited enhanced secretion above background that was statistically significant. Levels of GFP-GPI mannoprotein secretion was significantly depressed for forty of the mutants as compared to wild type. Proteins encoded by representative S. cerevisiae mutant strains identified in the screen are listed in Table 2. More specifically, these proteins correspond to mutant S. cerevisiae strains, which cause aberrant secretion of GFP-reporter protein. The data indicate that the assay method of the present invention is an efficient and sensitive means for differentiating mutant strains based on the secretion of the GFP cell wall marker protein.

TABLE 2

| | |
|---|---|
| PROTEIN | FUNCTION |

Mutant strains that hyper-secrete GFP reporter protein:

| | |
|---|---|
| TIR3 | Cell wall mannoprotein of Srp1/Tip1 family of serine alanine rich proteins |
| PBL2 | Phospholipase B involved in lipid metabolism |
| LIN1 | Component of U5 SnRNP which plays a role in chromosome segregation, |
| | mRNA splicing and DNA replication |

Mutant strains that hypo-secrete GFP reporter protein:

| | |
|---|---|
| DFG5 | Putative mannosidase, essential glycosyl phosphatidylinositol (GPI)-anchored |
| | membrane protein required for cell wall biogenesis in bud formation, involved |
| | in filamentous growth, homologous to Dcw1p |
| PAU2 | Part of 23-member seripauperin multigene family encoded mainly in |
| | subtelomeric regions, active during alcoholic fermentation, regulated by |
| | anaerobiosis, negatively regulated by oxygen, repressed by heme |
| DCW1 | Putative mannosidase, GPI anchored membrane protein required for cell wall |
| | biosynthesis in bud formation; homologous to Dfg5p |
| P1R1 | O-glycosylated protein required for cell wall stability; attached to the cell wall |
| | via beta-1,3-glucan; mediates mitochondrial translocation of Apn1p; |
| | expression regulated by the cell integrity pathway and by Swi5p during the cell |
| | cycle |

### Example 4

### High-Throughput Detection of Secreted GFP-Adhesin by Immunoblot Analysis

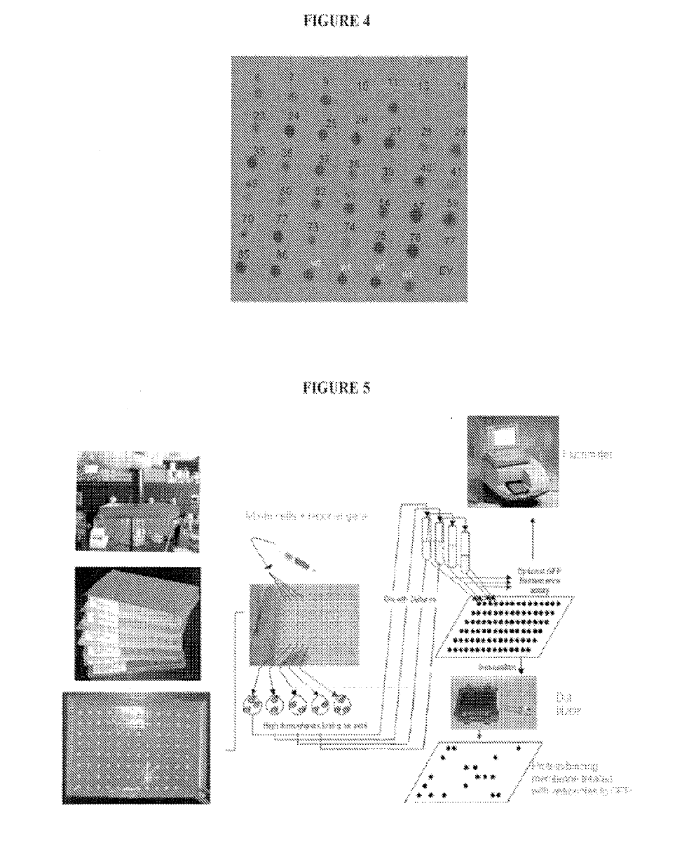

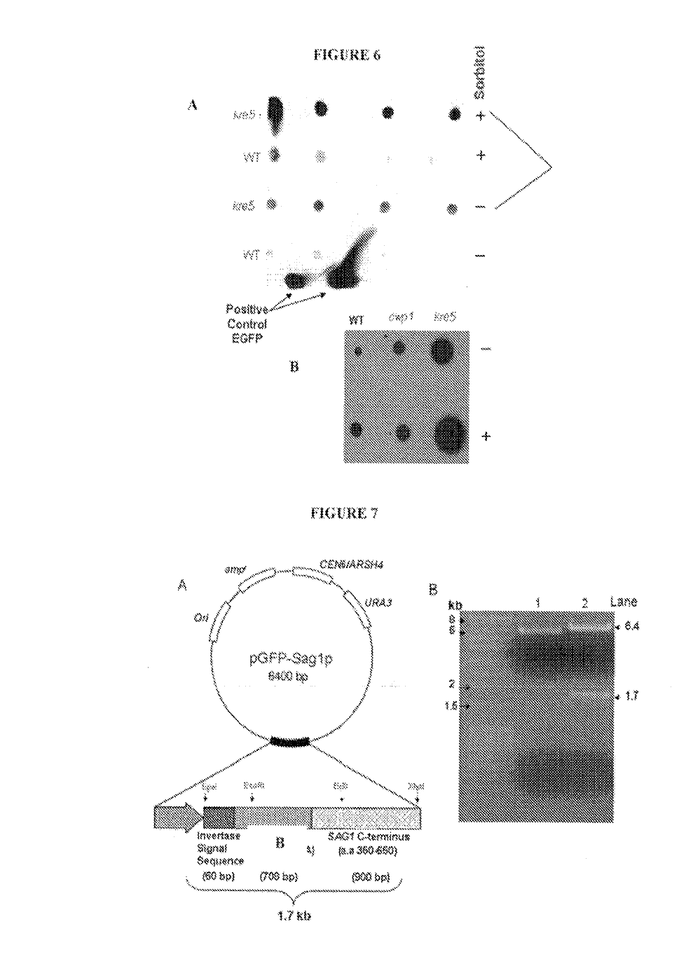

Cells were cultured and transformed as in Example 2. Replicates of each mutant sample were cultured in parallel and were grown to similar concentrations. Cell concentration was determined by reading the optical dens of the cell culture directly from the test tube using a spectrophotometer. The cell suspension was centrifuged for 10 minutes in order to pellet cells away from the supernatant. The supernatant was then collected and levels of the secreted GFP-mannoprotein was measured by immunoblot analysis. An aliquot of supernatant from each sample was transferred onto nitrocellulose and quantified by immunoblotting with a commercial antibody to GFP (FIG. 4).

All references cited and/or discussed in this specification are incorporated herein by reference in their entireties and to the same extent as if each reference was individually incorporated by reference.

We claim:

1. A method for detecting reporter-modified glycoprotein secreted from cells in culture, comprising the steps of:
(a) obtaining a plurality of cells that express one or more GPI-anchored proteins;
(b) transforming the cells in parallel with a plasmid expression vector comprising a gene sequence encoding a reporter-modified glycoprotein, wherein the reporter is a signal generating compound;
(c) incubating the transformed cells in culture media containing one or more osmoprotectants;
(d) isolating the culture media from the incubated cells; and
e) detecting reporter-modified glycoprotein secreted into the isolated culture media.

2. The method of claim 1, wherein one or more cells are derived from as fungal species.

3. The method of claim 2, wherein the fungal species is within a genus selected from Candida, Aspergillus, Ustillago, Cryptococcus, and Schizosaccharomyces.

4. The method of claim 1, wherein the reporter is GFP.

5. The method of claim 1, wherein the cells are incubated at a temperature of 15° C. to 20° C. for 1-3 days.

6. The method of claim 1, wherein the osmoprotectant is sorbitol.

7. The method of claim 1, wherein the plasmid expression vector is p416MG3.

8. The method of claim 1, wherein the glycoprotein is selected from a GPI-mannoprotein and an adhesin.

9. The method of claim 1, further comprising the step of measuring the amount of detected reporter-modified glycoprotein and/or dissociated reporter.

10. The method of claim 9, wherein the amount of detected reporter-modified glycoprotein and/or dissociated reporter is measured by a method selected from fluorimetry, immunoblot analysis, and a combination thereof.

11. A method for identifying genes required for GPI anchor-mediated attachment of a glycoprotein to the surface of cells in culture, comprising the steps of
(a) obtaining a plurality of cells, each containing a different gene deletion;
(b) transforming the cells in parallel with a plasmid expression vector comprising a gene sequence encoding a reporter-modified glycoprotein, wherein the reporter is a signal generating compound;
(c) incubating the transformed cells in culture media containing one or more osmoprotectants;
(d) isolating the culture media from the incubated cells;
(e) detecting reporter-modified glycoprotein secreted into the isolated culture media; and
(f) identifying genes required for GPI anchor-mediated attachment based on the amount of reporter-modified glycoprotein detected.

12. The method of claim 11, wherein one or more cells are derived from at fungal species.

13. The method of claim 11, wherein the reporter is GFP.

14. The method of claim 11, wherein the cells are incubated at a temperature of 15° C. to 20° C. for 1-3 days.

15. The method of claim 11, wherein the osmoprotectant is sorbitol.

16. A method for identifying chemical inhibitors of proteins that promote GPI anchor-mediated attachment of a glycoprotein to the surface of cells in culture, comprising the steps of;
(a) obtaining a plurality of cells;
(b) transforming the cells in parallel with a plasmid expression vector comprising a gene sequence encoding a reporter-modified glycoprotein, wherein the reporter is a signal generating compound;
(c) combining the transformed cells with culture media containing one or more osmoprotectants, and adding a different known chemical inhibitor to each cell culture;
(d) incubating the cell cultures;
(e) isolating the culture media from the incubated cells;
(f) detecting reporter-modified glycoprotein secreted into the isolated culture media; and
(g) identifying chemical inhibitors of proteins that promote GPI anchor-mediated attachment of a glycoprotein to the surface of cell in culture based on the amount of reporter-modified glycoprotein detected.

17. The method of claim 16, wherein one or more cells are derived from a fungal species.

18. The method of claim 16, wherein the reporter is GFP.

19. The method of claim 16, wherein the cells are incubated at a temperature of 15° C. to 20° C. for 1-3 days.

20. The method of claim 16, wherein the osmoprotectant is sorbitol.


SEQUENCE LISTING (6 sequences)

SEQ ID NO: 1 - DNA, 51 bp, Artificial Sequence
  Synthetic oligonucleotide
gcgccgactagtatgcttttgcaagctttccttttccttttggctggtttt

SEQ ID NO: 2 - DNA, 53 bp, Artificial Sequence
  Synthetic Oligonucleotide
cgccgggaattctgatgctgatattttagctgcaaaaccagccaaaaggaaaa

SEQ ID NO: 3 - DNA, 30 bp, Artificial Sequence
  Synthetic Oligonucleotide
gcgccggaattcagtaaaggagaagaactt

SEQ ID NO: 4 - DNA, 30 bp, Artificial Sequence
  Synthetic Oligonucleotide
cgccgcagatctgtatagttcatccatgcc

SEQ ID NO: 5 - DNA, 31 bp, Artificial Sequence
  Synthetic Oligonucleotide
gcggcgagatctagtgcggtattccactgga

SEQ ID NO: 6 - DNA, 30 bp, Artificial Sequence
  Synthetic Oligonucleotide
cgccgcctcgagttagaatagcaggtacga